(19) United States
(12) Patent Application Publication
(10) Pub. No.: US 2015/0374399 A1
(43) Pub. Date: Dec. 31, 2015

(54) Apparatus for Controlled Thinning of Biological Tissue and Method of Use Thereof

(71) Applicant: BACTERIN INTERNATIONAL, INC., Belgrade, MT (US)

(72) Inventors: Darrel Holmes, Bozeman, MT (US); Craig Smith, Bozeman, MT (US); Dustin B. Byrd, Bozeman, MT (US); Daniel Cox, Belgrade, MT (US); Helena M. Lovick, Belgrade, MT (US)

(73) Assignee: BACTERIN INTERNATIONAL, INC., Belgrade, MT (US)

(21) Appl. No.: 14/752,568

(22) Filed: Jun. 26, 2015

Related U.S. Application Data
(60) Provisional application No. 62/017,767, filed on Jun. 26, 2014.

(51) Int. Cl. A61B 17/322; B26D 1/147; B26D 7/02; G01N 1/06

(52) U.S. Cl. CPC A61B 17/322; G01N 1/06; B26D 1/147; B26D 7/02; A61B 2017/3225

(57) ABSTRACT

The disclosed invention includes an apparatus for cutting biological tissue in a substantially uniform manner and method for using the same.

### CROSS REFERENCE TO RELATED APPLICATIONS

This application claims priority and the benefit under 35 U.S.C. §119(e) to U.S. Provisional Patent Application Ser. No. 62/017,767 filed Jun. 26, 2014, which is incorporated herein in its entirety by reference.

### FIELD OF THE INVENTION

The present invention generally pertains to a slicing apparatus for reliable, pre-defined thinning of biological tissue.

### BACKGROUND AND DESCRIPTION OF THE RELATED ART

Instruments for slicing biological tissue are known in the prior art, however, many of these instruments are disclosed with various modifications to allow slicing of foodstuffs. Other instruments are hand-held, limiting the preciseness and repeatability of the resultant thinning of the biological tissue. However, the majority of these instruments are not capable of thinning biological tissue at a reliable, pre-defined thickness.

U.S. Pat. No. 3,613,754 entitled “Food Slicing Machine,” which is incorporated by reference in its entirety, discloses a gravity feed slicing machine with a cross feed mechanism to convert meat into thin slices of uniform thickness. U.S. Publication No. 2011/0177591 entitled “Method and Device for Producing Dermis,” which is incorporated by reference in its entirety, discloses a process for preparing dermis of a predefinable thickness comprising laying a section of skin onto a plate with a side of the skin directed away from the plate and removing material from said side of skin by means of a tool. Generally, the skin is connected to the plate by freezing and the excess skin is then removed by means of a milling cutter. U.S. Pat. No. 7,651,507 entitled “Tissue Processing System,” which is incorporated by reference in its entirety, discloses a tissue processing system including a tissue processor with a series of blades adjustable to remove donor tissue in the range of 250-1000 microns. The tissue processor is rotatable 90 degrees to produce uniform micrografts of tissue. Also disclosed is the general use of a cutting block to provide uniform thickness of the cuts of the tissue. U.S. Pat. No. 7,625,384 entitled “Method and Apparatus for Processing Dermal Tissue,” which is incorporated by reference in its entirety, discloses a portable and disposable device for processing harvested dermal tissue. The device is operable to slice harvested tissue into strips and further into fine particles.

A need remains for a cutting assembly capable of thinning biological tissue to a pre-defined, controllable thickness with precise thickness uniformity. It is desirable for the slicing apparatus to accommodate different biological tissue types and physical conditions (frozen and fresh tissue states) to provide controlled thinning of the tissue. Furthermore, it is desirable for the cutting surface to be openly accessible and rigidly stable to prevent flexing during the thinning of the biological tissues.

### SUMMARY OF THE INVENTION

The disclosed invention is directed to a slicing apparatus capable of thinning biological tissue to a pre-defined thickness. The slicing apparatus includes a rotary blade mounted beside a cutting plate with height adjustments to allow slicing of tissue to a predefined thickness. Additional optional features of the slicing apparatus are also disclosed. Optional features include, edge guides, exchangeable thickness adjustment tubes, and so forth.

Another aspect of the invention is a method for using the slicing apparatus. To use the slicing apparatus, the cutting plate is adjusted to a desired height by the regular spacing of thickness tubes. After the height of the cutting plate is set, the biological tissue to be thinned is placed on the cutting plate and then moved across the rotary blade for slicing.

An aspect of the invention is a slicing apparatus for thinning biological tissue to a predefined substantially uniform thickness. The apparatus includes a base structure, a motor-driven rotary blade mounted on the base structure, and a cutting plate with height adjustments. The cutting plate supports the biological tissue to be thinned at a selected height in relation to the motor driven rotary blade.

An aspect of the invention is a method for substantially uniform thinning of biological tissue to a predefined thickness using a slicing apparatus. The slicing apparatus includes a base structure, a motor-driven rotary blade mounted on the base structure, and a cutting plate. The cutting plate has a plurality of height adjustment tubes to support the biological tissue to be thinned. The cutting plate is at a selected height in relation to the motor driven rotary blade. The method for using the apparatus includes, setting the height of the cutting plate with a plurality of height adjustment tubes, positioning the biological tissue to be thinned downward on the cutting plate, rotating the rotary blade, and moving the biological tissue to be thinned across the rotating rotary blade until the biological tissue is cut to the desired length.

### BRIEF DESCRIPTION OF THE FIGURES

FIG. 1 illustrates a perspective, fully assembled view of the slicing apparatus of the present invention;

FIG. 2 illustrates an isometric, exploded view of the slicing apparatus of the present invention;

FIG. 3 illustrates a perspective, exploded side view of the slicing apparatus of the present invention;

FIG. 4 illustrates an isometric, exploded view of the base and base covers of the present invention;

FIG. 5 illustrates a right cylinder and a bent cylindrical tube; and

FIG. 6 illustrates a perspective, side view of the cutting plate mounted on bent cutting adjustment tubes.

### DETAILED DESCRIPTION OF THE INVENTION

The invention relates to a slicing apparatus and a method of using the apparatus.

An aspect of the invention is a slicing apparatus for substantially uniform thinning of a biological tissue to a predefined thickness. The apparatus includes a base structure, a mounted, motor-driven rotary blade, and a cutting plate with height adjustments to support the biological tissue to be thinned at a selected height.

An aspect of the invention is a slicing apparatus for thinning biological tissue. The apparatus includes a base structure, a motor-driven rotary blade mounted on the base structure, and a cutting plate with height adjustments. The cutting plate supports the biological tissue to be thinned at a selected height in relation to the motor driven rotary blade. The apparatus can thin or cut the biological tissue to a desired thickness which can be predetermined. In particular, the thickness can be substantially uniform.

The plane of the cutting plate height level can be adjusted with a plurality of height adjustment structures, such as tubes. The tubes can be different lengths. For example, the tubes can be between 0.01 mm to about 30 mm. In some embodiments, the tubes can be right cylinders, or bent cylinders, wherein the bent angle of the bent cylinders can be between about 90 degrees and about 180 degrees. In some embodiments, the bent angle can be about 90 degrees, about 100 degrees, about 110 degrees, about 120 degrees, about 130 degrees, about 140 degrees, about 150 degrees, about 160 degrees, about 170 degrees, or about 180 degrees. The tubes can be made of a rigid material. Suitable materials include, but are not limited to, metals, thermoplastics, carbides and ceramics. The cutting plate can be held rigidly in place with the tubes in order to prevent movement of the cutting plate during use. The cutting plate can be smooth, or can include groves or other textures such as ridges, dots or other raised or depressed structures.

The blade can be driven by any suitable motor, including but not limited to, a pneumatic motor or an electric motor. The motor can operate between about 0.1 hp and about 5 hp and between about 50 rpm to about 1000 rpm. The blade can be made of any suitable material including, but not limited to, metals, thermoplastics, carbides and ceramics.

The entire apparatus, or parts of the apparatus, can be autoclaved for cleaning and achieving or maintaining sterility. The autoclave can operate at temperatures between about 80° C. and about 200° C., and a pressure of between about 0.01 atm and about 2 atm, for between about 2 minutes and about 45 minutes.

The position of the cutting plate relative to the slicing apparatus can be adjusted. A plane of the cutting plate can be positioned at a substantially uniform height. In some embodiments, the cutting plate can be parallel to the plane of the motor-driven rotary blade. The plane of the cutting plate can be positioned at an angle between about 0 degrees and about 90 degrees in relation to a plane of the rotary blade. In some embodiments, the plane of the cutting plate can be position at an angle of about 0 degrees, about 10 degrees, about 20 degrees, about 30 degrees, about 40 degrees, about 50 degrees, about 60 degrees, about 70 degrees, about 80 degrees, or about 90 degrees in relation to a plane of the rotary blade. The cutting plate can also include edge guides to assist in the placement of the biological tissue with relation to the rotary blade. The guides can have attachments to attach the tissue to be thinned to the guide. Furthermore, in some embodiments, the biological tissue can be moved across the rotating rotary blade such that a thinned biological tissue has a substantially uniform height throughout the length of the thinned biological tissue. The biological tissue to be thinned can be manually moved across the rotating rotary blade, or can be automatically moved across the rotating rotary blade, such as by a motorized movement of the cutting plate in relation to the blade.

In some embodiments, a separate flat plate can be used. The biological tissue can be attached to a separate flat plate, which is then placed onto the cutting plate or attached to the cutting apparatus. The manner of attaching the biological tissue to a separate flat surface can include, but is not limited to, vacuum suction, freezing, gripping teeth, friction patterns, or combinations thereof.

The biological tissue can be held in place on the cutting plate with an attachment mechanism. The attachment mechanism can be at least one device that holds the tissue in place, including but not limited to, a gripper, a tong, a bracket, a pincer, a plate, and combinations of the attachment elements. In some embodiments, the biological tissue to be thinned can be attached to the cutting plate.

Any suitable biological tissue can be sliced using the apparatus. Suitable biological tissues include, but are not limited to, allograft tissue, autograft tissue, xenograft tissue, cortical bone, cancellous bone, demineralized bone, connective tissue, tendon, pericardium, dermis, cornea, dura matter, fascia, heart valve, ligament, capsular graft, cartilage, collagen, nerve, placental tissue, and combinations thereof. In some embodiments, the tissue can be human allograft. In some embodiments, the tissue can be human dermis. The tissue can be treated prior to thinning, for example by demineralizing or partially demineralizing the tissue, seeded with a material or other suitable treatment. Furthermore, the biological tissues can be thinned in a frozen condition, a chilled condition, a room temperature condition, a warmed condition, or combinations thereof. The thinning of the biological tissues can take place at with the temperature of the biological tissue ranging from about −80° C. to +40° C., or at any intermediate temperature or range within these end points. In some embodiments, the biological tissue can be at a temperature of about −80° C., about −70° C., about −60° C., about −50° C., about −40° C., about −30° C., about −20° C., about −10° C., about 0° C., about 10° C., about 20° C., about 30° C., or about 40° C. In some embodiments, the biological tissue can be fully frozen prior to the thinning process. The biological tissue can be maintained at a frozen or low temperature throughout the cutting process. In some embodiments, the biological tissue can be frozen in a controlled manner to minimize tissue damage. The biological tissue can be frozen after treatment with a cryoprotectant. Suitable cryoprotectants include, but are not limited to, aqueous solutions containing alcohols, dimethyl sulfoxide, dextrans, polyvinylpyrrolidones, sugars, amino acids, diols, and combinations thereof. In some embodiments, the blade can be heated during use to facilitate the ease of thinning frozen tissue.

In some embodiments, the biological tissue can be in a solidified material, such as a solidified fluid, at the time it is contacted with a blade for thinning or cutting of the tissue. The solidified fluid can be an aqueous fluid, a polymer, a wax, a gel or other material that can support or stabilize the tissue. Suitable aqueous fluids include, but are not limited to, water and saline. The aqueous fluid can include at least one buffer to promote freezing conditions. Freezing conditions include, but are not limited to, flash freezing at cryogenic temperatures, slow freezing at about 0° C., and deep freezing at about −80° C. In some embodiments, the freezing conditions result in a substantially flat orientation of the biological tissue within the solidified material. Suitable buffers include, but are not limited to, phosphate buffered salines, Hank's balanced salt solutions, and Tris-HCl buffers. By way of example only, the biological tissue can be contained within an ice block. The solidified fluid with the biological tissue can then be placed on the cutting plate or the separate plate for thinning or cutting of the tissue. By way of example, the ice block can be attached to a cutting plate or the separate plate. In a preferred embodiment, the tissue can be contacted with a liquid aqueous solution such as water or saline while in contact with the separate plate or cutting plate and frozen. The assembly of the plate, with the frozen tissue encased in frozen liquid, can then be used to position the tissue for cutting in the apparatus of the present invention. Treatment of a biological tissue within a solidified fluid can achieve highly uniform thicknesses of the thinned biological tissue and/or allow for more efficient use of such materials by allowing for the harvesting of multiple cut pieces of tissue from a single piece of starting material because of reduced damage in the cutting process. Additionally, cutting the tissue while in the solidified state reduces drag on the cutting blade during slicing compared to methods that do not use a solidified state during cutting. The reduced drag on the cutting blade during the slices results in a thinned biological tissue with a smooth topology post slicing. After cutting, the thinned tissue can be removed from the solidified fluid by any suitable means, including for example, melting the solidified fluid into its liquid state.

The thinned tissues cut using this apparatus can provide tissue of thicknesses ranging from about 0.01 mm to about 30 mm, from about 0.2 mm to about 15 mm, from about 0.3 mm to about 10 mm, or from about 0.5 mm to about 5 mm. The biological tissues cut using this method can provide tissue of lengths ranging from about 5 mm to about 300 mm, from about 10 mm to about 250 mm, or from about 15 mm to about 225 mm. The biological tissues cut using this apparatus can yield a sliced tissue with an angle between the cut on surface and an uncut surface of the tissue of about 0 to about 90 degrees. The tolerance on the thickness of a thinned tissue can be between about 0.001 mm to about 1 mm. In some embodiments, the tolerance can be about 0.001 mm, about 0.01 mm, about 0.1 mm, about 0.2 mm, about 0.5 mm, or about 1 mm.

A blade guard can also be included on the apparatus. In some embodiments, the apparatus can include a catch pan and/or a drip pan. The catch pan can collect the thinned material, while the drip pan can collect particles generated during the thinning process.

An aspect of the invention is a method for substantially uniform thinning of biological tissue to a predefined thickness using a slicing apparatus. The slicing apparatus includes a base structure, a motor-driven rotary blade mounted on the base structure, and a cutting plate. The cutting plate has a plurality of height adjustment structures, such as tubes or other supporting materials, tubes to support the biological tissue to be thinned. The cutting plate is at a selected height in relation to the motor driven rotary blade. The method for using the apparatus includes, setting the height of the cutting plate with a plurality of height adjustment tubes, positioning the biological tissue to be thinned downward on the cutting plate, rotating the rotary blade, and moving the biological tissue to be thinned across the rotating rotary blade until the biological tissue is cut to the desired length.

The plane of the cutting plate height level can be adjusted with a plurality of height adjustment tubes. The tubes can be available in different lengths. For example, the tubes can be between 0.01 mm to about 30 mm. In some embodiments, the tubes can be right cylinders, or bent cylinders, wherein the bent angle of the bent cylinders can be between about 90 degrees and about 180 degrees. In some embodiments, the bent angle can be about 90 degrees, about 100 degrees, about 110 degrees, about 120 degrees, about 130 degrees, about 140 degrees, about 150 degrees, about 160 degrees, about 170 degrees, or about 180 degrees. The tubes can be made of a rigid material. Suitable materials include, but are not limited to, metals, thermoplastics, carbides and ceramics. The cutting plate can be held rigidly in place with the tubes in order to prevent movement of the cutting plate during use. The cutting plate can be smooth, or can include groves or other textures such as ridges, dots or other raised or depressed structures.

The blade can be driven by any suitable motor, including but not limited to, a pneumatic motor or an electric motor. The motor can operate between about 0.1 hp and about 5 hp and between about 50 rpm to about 1000 rpm. The blade can be made of any suitable material including, but not limited to, metals, thermoplastics, carbides and ceramics.

The entire apparatus, or parts of the apparatus, can be autoclaved for cleaning and achieving or maintaining sterility. The autoclave can operate at temperatures between about 80° C. and about 200° C., and a pressure of between about 0.01 atm and about 2 atm, for between about 2 minutes and about 45 minutes.

The position of the cutting plate relative to the slicing apparatus can be adjusted. A plane of the cutting plate can be positioned at a substantially uniform height. In some embodiments, the cutting plate can be parallel to the plane of the motor-driven rotary blade. The plane of the cutting plate can be positioned at an angle between about 0 degrees and about 90 degrees in relation to a plane of the rotary blade. In some embodiments, the plane of the cutting plate can be position at an angle of about 0 degrees, about 10 degrees, about 20 degrees, about 30 degrees, about 40 degrees, about 50 degrees, about 60 degrees, about 70 degrees, about 80 degrees, or about 90 degrees in relation to a plane of the rotary blade. The cutting plate can also include edge guides to assist in the placement of the biological tissue with relation to the rotary blade. Furthermore, in some embodiments, the biological tissue can be moved across the rotating rotary blade such that a thinned biological tissue has a substantially uniform height throughout the length of the thinned biological tissue. The biological tissue to be thinned can be manually moved across the rotating rotary blade, or can be automatically moved across the rotating rotary blade, such as by a motorized movement of the cutting plate in relation to the blade.

The biological tissue can be held in place on the cutting plate with an attachment mechanism. The attachment mechanism can be at least one device that holds the tissue in place, including but not limited to, a gripper, a tong, a bracket, a pincer, a plate, and combinations of the attachment elements. In some embodiments, the biological tissue to be thinned can attached to the cutting plate.

Any suitable biological tissue can be sliced using the apparatus. Suitable biological tissues include, but are not limited to, allograft tissue, autograft tissue, xenograft tissue, cortical bone, cancellous bone, demineralized bone, connective tissue, tendon, pericardium, dermis, cornea, dura matter, fascia, heart valve, ligament, capsular graft, cartilage, collagen, nerve, placental tissue, and combinations thereof. In some embodiments, the tissue can be human allograft. In some embodiments, the tissue can be human dermis. Furthermore, the biological tissues can be thinned in a frozen condition, a chilled condition, a room temperature condition, a warmed condition, or combinations thereof. The thinning of the biological tissues can take place at with the temperature of the biological tissue ranging from about −80° C. to +40° C., or any intermediate range within those endpoints. In some embodiments, the biological tissue can be at a temperature of about −80° C., about −70° C., about −60° C., about −50° C., about −40° C., about −30° C., about −20° C., about −10° C., about 0° C., about 10° C., about 20° C., about 30° C., or about 40° C. In some embodiments, the biological tissue can be fully frozen prior to the thinning process. The biological tissue can be maintained at a frozen or low temperature throughout the cutting process. In some embodiments, the biological tissue can be frozen in a controlled manner to minimize tissue damage. The biological tissue can be frozen after treatment with a cryoprotectant. Suitable cryoprotectants include, but are not limited to, aqueous solutions containing alcohols, dimethyl sulfoxide, dextrans, polyvinylpyrrolidones, sugars, amino acids, diols, and combinations thereof. In some embodiments, the blade can be heated during use to facilitate the ease of thinning frozen tissue

In some embodiments, the biological tissue can be in a solidified material, such as a solidified fluid, at the time it is contacted with a blade for thinning or cutting of the tissue. The solidified fluid can be an aqueous fluid, a polymer, a wax, a gel or other material that can support or stabilize the tissue. Suitable aqueous fluids include, but are not limited to, water and saline. The aqueous fluid can include at least one buffer to promote freezing conditions. Freezing conditions include, but are not limited to, flash freezing at cryogenic temperatures, slow freezing at about 0° C., and deep freezing at about −80° C. In some embodiments, the freezing conditions result in a substantially flat orientation of the biological tissue within the solidified material. Suitable buffers include, but are not limited to, phosphate buffered salines, Hank's balanced salt solutions, and Tris-HCl buffers. By way of example only, the biological tissue can be contained within an ice block. The solidified fluid with the biological tissue can then be placed on the cutting plate or the separate plate for thinning or cutting of the tissue. By way of example, the ice block can be attached to a cutting plate or the separate plate. In a preferred embodiment, the tissue can be contacted with a liquid aqueous solution such as water or saline while in contact with the separate plate or cutting plate and frozen. The assembly of the plate, with the frozen tissue encased in frozen liquid, can then be used to position the tissue for cutting in the apparatus of the present invention. Treatment of a biological tissue within a solidified fluid can achieve highly uniform thicknesses of the thinned biological tissue and/or allow for more efficient use of such materials by allowing for the harvesting of multiple cut pieces of tissue from a single piece of starting material because of reduced damage in the cutting process. Additionally, cutting the tissue while in the solidified state reduces drag on the cutting blade during slicing compared to methods that do not use a solidified state during cutting. The reduced drag on the cutting blade during the slices results in a thinned biological tissue with a smooth topology post slicing. After cutting, the thinned tissue can be removed from the solidified fluid by any suitable means, including for example, melting the solidified fluid into its liquid state.

The thinned tissues cut using this apparatus can provide tissue of thicknesses ranging from about 0.01 mm to about 30 mm, from about 0.2 mm to about 15 mm, from about 0.3 mm to about 10 mm, or from about 0.5 mm to about 5 mm. The biological tissues cut using this method can provide tissue of lengths ranging from about 5 mm to about 300 mm, from about 10 mm to about 250 mm, or from about 15 mm to about 225 mm. The biological tissues cut using this apparatus can yield a sliced tissue with an angle between the cut on surface and an uncut surface of the tissue of about 0 to about 90 degrees. The tolerance on the thickness of a thinned tissue can be between about 0.001 mm to about 1 mm. In some embodiments, the tolerance can be about 0.001 mm, about 0.01 mm, about 0.1 mm, about 0.2 mm, about 0.5 mm, or about 1 mm.

A blade guard can also be included on the apparatus. In some embodiments, the apparatus can include a catch pan and/or a drip pan. The catch pan can collect the thinned material, while the drip pan can collect particles generated during the thinning process.

The thinned tissues cut using this method can provide tissue of thicknesses ranging from about 0.1 mm to about 30 mm, from about 0.2 mm to about 15 mm, from about 0.3 mm to about 10 mm, or from about 0.5 mm to about 5 mm. The biologics tissues cut using this method can provide tissue of lengths ranging from about 5 mm to about 300 mm, from about 10 mm to about 250 mm, or from about 15 mm to about 225 mm. The biological tissues cut using this method can yield a sliced tissue with an angle between the cut on surface and an uncut surface of the tissue of about 0 to about 90 degrees.

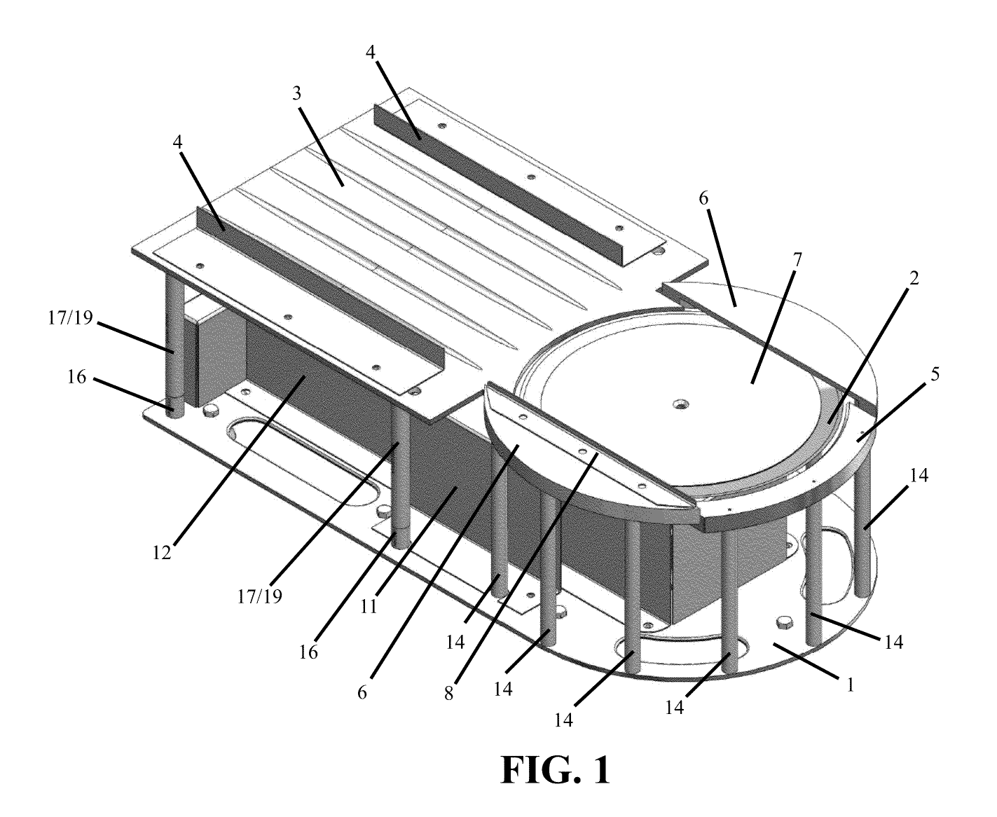

Embodiments of the invention and their advantages are best understood by referring to FIGS. 1 through 6 of the drawings, like numerals being used for like and corresponding parts of the various drawings.

FIG. 1 illustrates a fully assembled view of the slicing apparatus of the invention. The slicing apparatus is comprised of a base 1, rotary cutting blade 2, and a cutting plate 3. The cutting plate 3 and the blade 2 edge are exposed to allow introduction of multiple tissue sizes to the cutting surface. Additionally, the design of the cutting plate 3 and the exposure of the blade 2 edge allow the use of multiple styles and sizes of tissue holders during the thinning process. Tissue holders can consist of grippers, tongs, brackets, pincers, plates, combinations thereof and other implements connected in some manner to the biological tissue to be thinned.

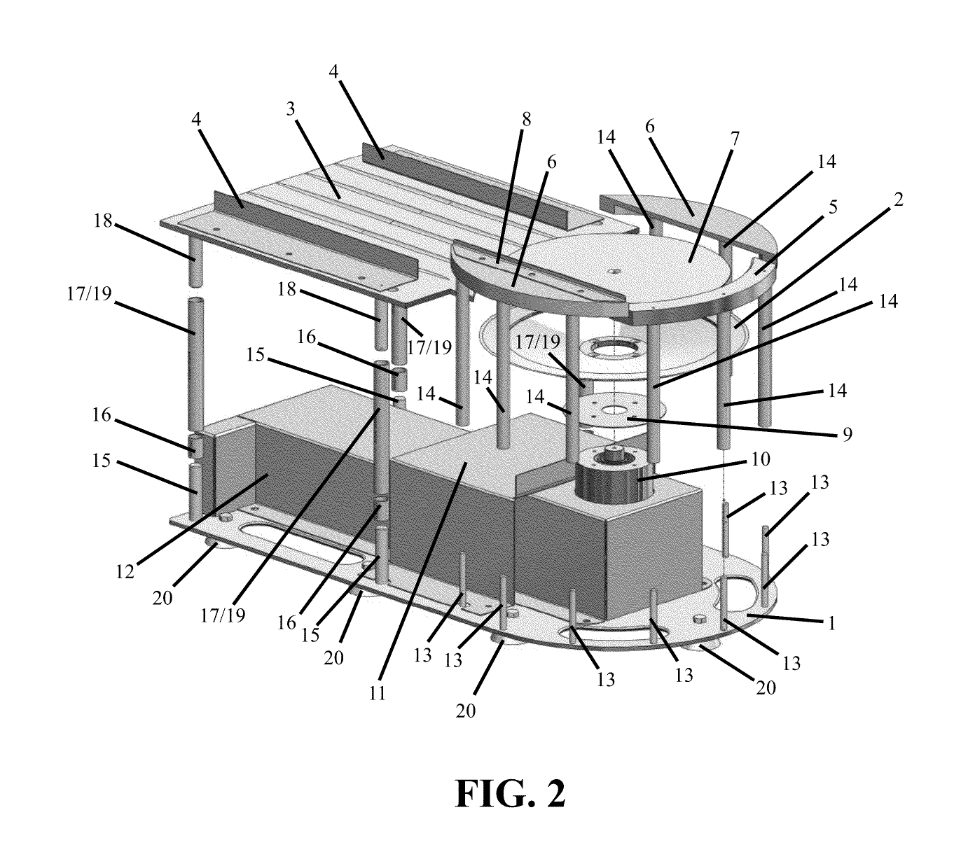

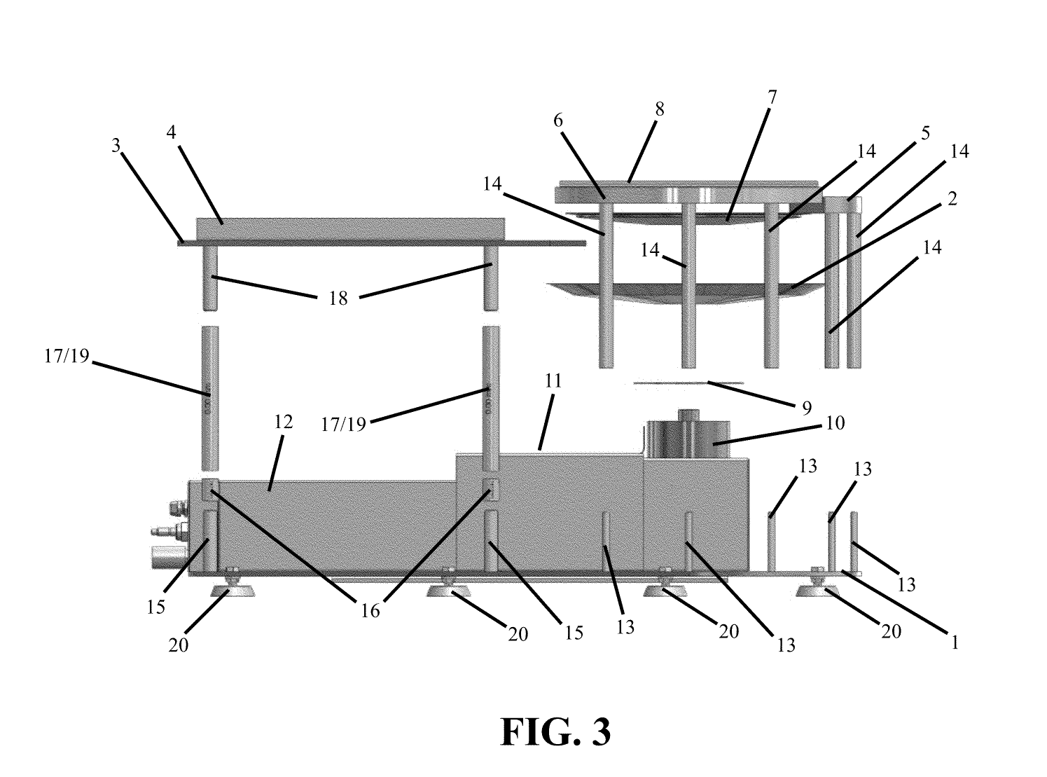

The cutting plate can have cutting guides 4 to assist in positioning an article to be cut on the cutting plate 3. The cutting plate 3 can have a smooth or grooved surface. The cutting guides 4 can have brackets to allow attachment of the article to be cut onto the cutting plate 3. The cutting guides 4 can be adjustable to accommodate variously sized articles to be cut. The cutting guides 4 can be removable. As illustrated in FIG. 2, multiple blade guards can be mounted onto guard posts 13 on the base 1 over the blade 2, such as an end guard 5 and edge guards 6. An additional center guard 7 can be mounted over the blade 2, and under the blade guards 5 and 6. The center guard 7 can have a smooth or grooved surface. The center guard 7 can have attached edge guides. The edge guard 6 can include an edge guide 8 to orient the article to be cut over the blade 2. As illustrated in the exploded view of FIG. 2 and in the side view of FIG. 3, a drip guard 9 can be mounted under the blade 2 onto the hub 10 protruding from the drive shaft 21. A catch pan 11 can be positioned under the blade 2 to provide a platform for collection of the thinned tissue. The drive train can be protected by a drive train cover 12. The catch pan 11 can extend the over a part or all of the drive train cover 12. The blade guards 5 and 6 can be attached to posts 14 to securely hold the guards in place around the blade 2.

The mounting and orientation of the cutting plate 3 determines the shape and thickness of tissue sliced with the slicing apparatus of the present invention. In order to adjust the height of the cutting plate to align with the top of the blade 2, at least four zeroing tubes 16 of a pre-determined height are mounted onto an equal number of tube posts 15 on the slicing apparatus' base 1. Then an equal number of thickness adjustment tubes 17 of a set length are placed onto the tube posts 15 above the zeroing tubes 16. The cutting plate is mounted into the thickness adjustment tubes 17 through an equal number of attached cutting plate posts 18. The zero tubes 16 and thickness adjustment tubes 17 are right cylinders of a height established to align or “zero” the cutting plate 3 top surface with the top surface of the blade 2. If the height of the cutting plate 3 is not zeroed with respect to the height of the blade 2, the zeroing tubes can be switched out with tubes of a slightly different height (such as about +0.2 mm, about +0.1 mm, about −0.1 mm, about −0.2 mm, etc.). After the cutting plate 3 is zeroed in relation to the plane of the blade's surface, the height of the cutting plate 3 can be lowered by removing the thickness adjustment tubes 17 and replacing the tubes with cutting adjustment tubes 19. The cutting adjustment tubes 19 can be right cylinders of lengths slightly less than the thickness adjustment tubes 17 used to zero the cutting plate 3 position with respect to the blade 2 height. For example, the lengths of the cutting adjustment tubes 19 can be about 1.50 mm or about 0.75 mm shorter than the thickness adjustment tubes 17 used to zero the cutting plate 3 position. In some embodiments, the height of the cutting plate 3 can be adjusted manually or electronically by telescoping posts or other equivalent means.

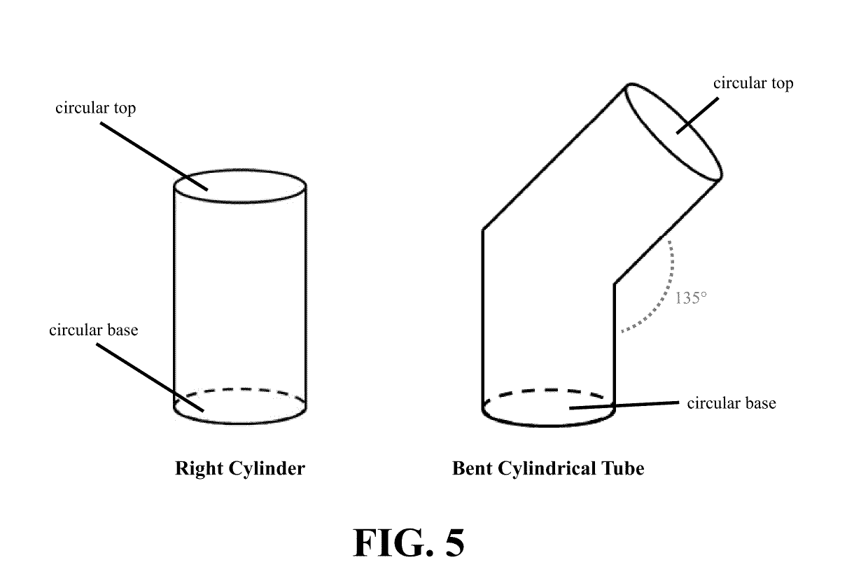

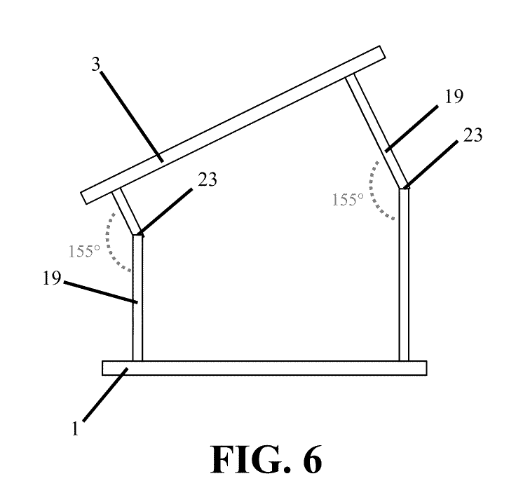

If an angled cutting surface is desired, the cutting adjustment tubes 19 can be bent at an angle between about 90 to about 180 degrees. FIG. 5 provides an illustration of a bent cylindrical tube bent to about 135 degrees, such as of the shape for a cutting adjustment tube 19 bent to about 135 degrees. As illustrated in FIG. 6, in another embodiment the cutting adjustment tubes 19 can be flexible, hinged, or bendable 23 in some manner to allow the user to alter the angle of the cutting plate 3. If bent cutting adjustment tubes 19 are used, one side of the cutting plate 3 can be lower than the other side. This need for differing heights of the sides of the cutting plate 3 can be accommodated by using different lengths of the bent cutting adjustment tubes 19 on the two different sides of the cutting plate 3. This would allow the cutting plate 3 to be held securely at the desired angle.

The cutting apparatus can be placed on foot mounts 20. The foot mounts 20 can be composed of vibration dampening materials such as rubber. Additionally the foot mounts 20 can have height adjusting features such as a screw-in base to allow the user to adjust the levelness of the apparatus' base 1.

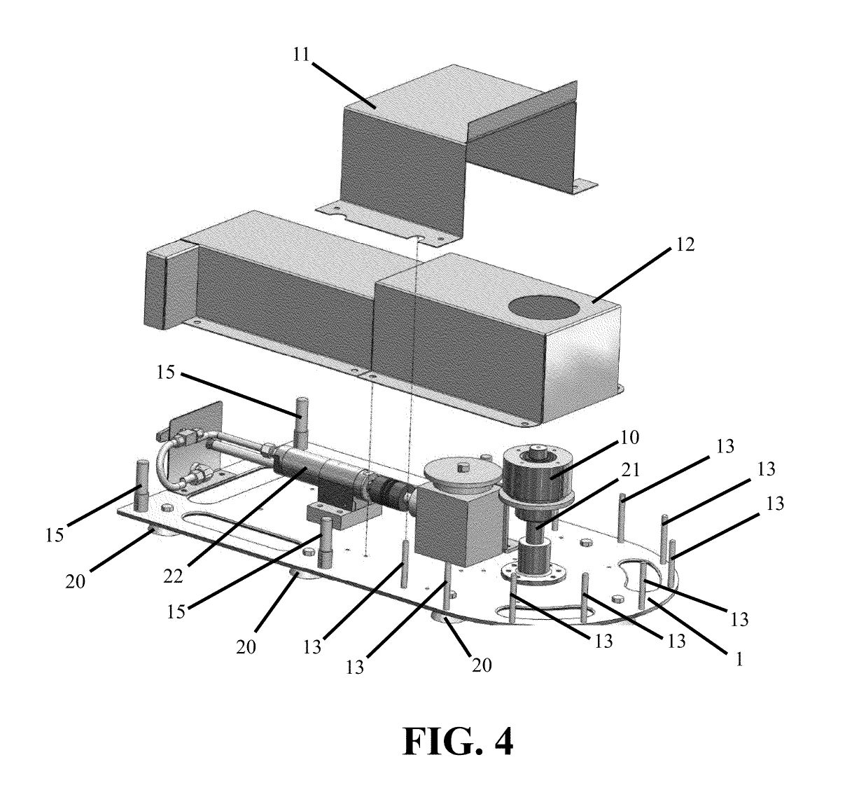

As illustrated in FIG. 4, the assembly can be fully disassembled from the base mount. The drive train cover 12 is the last item removed for routine disassembly. The ability to disassemble the slicing apparatus facilitates cleaning and sterilization of the disassembled parts. The parts of the slicing apparatus removed from the base 1 can be composed of heat stable materials to allow steam sterilization or autoclaving of the assembly. The motor 22 can be partially disassembled or replaced as needed for repairs and cleaning.

The slicing assembly can be made from surgical grade materials, including surgical steel, aluminum, treated aluminum, treated steel, aluminum alloys, steel alloys, polymers, and combinations thereof.

The biological tissues to be thinned can consist of allograft tissue, autograft tissue, xenograft tissue, or combinations thereof. In preferred embodiments, the biological tissue to be thinned is human allograft tissue. The biological tissue to be thinned can be comprised of cortical bone, cancellous bone, demineralized bone, connective tissue, tendon, pericardium, dermis, cornea, dura matter, fascia, heart valve, ligament, capsular graft, cartilage, collagen, nerve, placental tissue, and combinations thereof. By way of example, demineralized bone can be produced using the methods discussed in U.S. Pat. No. 8,574,825, which is incorporated by reference in its entirety. In a preferred embodiment, the biological tissue to be thinned is human dermis. Furthermore, the biological tissues can be thinned in a frozen condition, a chilled condition, a room temperature condition, a warmed condition, or combinations thereof. The thinning of the biological tissues can take place at with the temperature of the biological tissue ranging from about −80° C. to +40° C. In some embodiments, the biological tissue can be fully frozen prior to the thinning process. The biological tissue can be fully frozen within an ice block prior to the thinning process. The biological tissue can be fully frozen within an ice block during the thinning process. Suitable aqueous-based solutions may be used to suspend and contain the biological tissue as frozen within an ice block, these include, but are not limited to, water, salines, buffers, and balanced salt solutions. The biological tissue can be maintained at a frozen or low temperature throughout the cutting process. In some embodiments, the biological tissue can be frozen in a controlled manner to minimize tissue damage. The biological tissue can be frozen after treatment with a cryoprotectant. Suitable cryoprotectants include, but are not limited to, aqueous solutions containing alcohols, dimethyl sulfoxide, dextrans, polyvinylpyrrolidones, sugars, amino acids, diols, and combinations thereof. In some embodiments, the blade 2 can be heated during use to facilitate the ease of thinning frozen tissue.

Another embodiment of the present disclosure is a method of thinning biological tissue to a pre-defined thickness using the cutting assembly. The biological tissues cut using this method can be thinned to a substantially uniform thickness. The biological tissues cut using this method can be thinned to a substantially uniform, angled thickness. The biological tissues cut using this method can provide tissue of thicknesses ranging from about 0.1 mm to about 30 mm, from about 0.2 mm to about 15 mm, from about 0.3 mm to about 10 mm, or from about 0.5 mm to about 5 mm. The biologics tissues cut using this method can provide tissue of lengths ranging from about 5 mm to about 300 mm, from about 10 mm to about 250 mm, or from about 15 mm to about 225 mm. The biological tissues cut using this method can yield a sliced tissue with an angle between the cut on surface and an uncut surface of the tissue of about 0 to about 90 degrees. In some embodiments, the biological tissues may be thinned multiple times during the cutting process to provide more than one slice of biological tissue thinned to a pre-defined thickness.

A method of thinning biological tissue to a pre-defined thickness using the disclosed cutting assembly can consist of adjusting the height of the cutting plate 3 to a predefined level using right cylinder-shaped, cutting adjustment tubes 19. Once the cutting plate 3 is at the desired height with respect to the blade 2 of the cutting apparatus, the biological tissue to be thinned can be placed directly onto the cutting plate 3 or directly attached to the cutting apparatus. In some embodiments, the biological tissue can be attached to a separate flat surface; the tissue so attached can then be placed onto the cutting plate 3 or attached to the cutting apparatus. The manner of attaching the biological tissue to a separate flat surface can include, but is not limited to, vacuum suction, freezing, gripping teeth, friction patterns, or combinations thereof. As the blade 2 rotates, the biological tissue to be thinned can be manually or mechanically moved across the blade 2 until the desired length of the cut is complete. The resultant thinned biological tissue has a pre-defined and substantially uniform thickness.

An alternative method of thinning biological tissue to a pre-defined thickness using the disclosed cutting assembly can consist of adjusting the height of the cutting plate 3 to a predefined level and angle using bent cutting adjustment tubes 19. Once the cutting plate 3 is at the desired height and angle with respect to the blade 2 of the cutting apparatus, the biological tissue to be thinned can be placed onto or attached to the cutting plate 3. As the blade 2 rotates, the biological tissue to be thinned can be manually or mechanically moved across the blade 2 until the desired length of the cut is complete. The resultant thinned biological tissue has a pre-defined angled thickness.

While the embodiments of the invention described herein have been characterized as being preferred, those of ordinary skill in the art will understand and appreciate the existence of variations, combinations, and equivalents of the specific embodiments, methods, and examples. The invention should therefore not be limited by the above described embodiments, methods, and examples, but by all embodiments and methods within the scope and spirit of the invention as claimed.

What is claimed is:

1. A slicing apparatus for thinning biological tissue to a predefined substantially uniform thickness, comprising:
a base structure;
a motor-driven rotary blade mounted on the base structure;
and a cutting plate with height adjustments to support the biological tissue to be thinned at a selected height in relation to the motor driven rotary blade.

2. The slicing apparatus of claim 1, where the biological tissue is contained within a solidified material attached to the cutting plate.

3. The slicing apparatus of claim 2, wherein the solidified material is an ice block.

4. The slicing apparatus of claim 3, where the ice block comprises of water.

5. The slicing apparatus of claim 4, where the water contains at least one buffer.

6. The slicing apparatus of claim 5, where the biological tissue is in a substantially flat orientation within the ice block.

7. The slicing apparatus of claim 5, where the at least one buffer is phosphate buffered saline.

8. The slicing apparatus of claim 6, where the substantially flat orientation provides the ability to obtain multiple slices of the biological tissue.

9. The slicing apparatus of claim 3, where the ice block maintains the biological tissue in a substantially frozen state during slicing.

10. The slicing apparatus of claim 9, where the substantially frozen state of the biological tissue results in reduced drag on the cutting blade during the slicing process.

11. The slicing apparatus of claim 10, where the reduced drag on the cutting blade during the slicing process results in a thinned biological tissue with a smooth topology post-slicing.

12. The slicing apparatus of claim 1, wherein a plane of the cutting plate height level is adjusted with a plurality of height adjustment tubes.

13. The slicing apparatus of claim 12, wherein the plurality of height adjustment tubes are composed of a rigid material.

14. The slicing apparatus of claim 12, wherein the plurality of height adjustment tubes are available in different lengths.

15. The slicing apparatus of claim 12, wherein the plurality of height adjustment tubes are right cylinders.

16. The slicing apparatus of claim 12, wherein the plurality of height adjustment tubes are bent cylinders.

17. The slicing apparatus of claim 16, where the plurality of height adjustment tubes are bent between about 90 degrees and about 180 degrees.

18. The slicing apparatus of claim 1, wherein the motor-driven rotary blade is driven by a motor selected from the group consisting of a pneumatic motor and an electric motor.

19. The slicing apparatus of claim 1, wherein the slicing apparatus is autoclavable.

20. The slicing apparatus of claim 1, wherein the selected height of the plane of the cutting plate is adjustable.

21. The slicing apparatus of claim 1, wherein a plane of the cutting plate is positioned at a substantially uniform height.

22. The slicing apparatus of claim 1, wherein a plane of the cutting plate is positioned parallel to the plane of the motor-driven rotary blade.

23. The slicing apparatus of claim 1, wherein a plane of the cutting plate is positioned at an angle between about 0 degrees and about 90 degrees in relation to a plane of the rotary blade.

24. The slicing apparatus of claim 1, wherein the cutting plate is held rigidly in place by a plurality of height adjustment tubes to prevent movement of the cutting plate during biological tissue thinning.

25. The slicing apparatus of claim 1, wherein a biological tissue is held in place on the cutting plate through an attachment mechanism, wherein the attachment mechanism is selected from the group consisting of grippers, tongs, brackets, pincers, plates, and combinations thereof.

26. The slicing apparatus of claim 1, wherein the biological tissue is selected from the group consisting of allograft tissue, autograft tissue, xenograft tissue and combinations thereof.

27. The slicing apparatus of claim 1, wherein the biological tissue is selected from the group consisting of cortical bone, cancellous bone, demineralized bone, connective tissue, tendon, pericardium, dermis, cornea, dura matter, fascia, heart valve, ligament, capsular graft, cartilage, collagen, nerve, placental tissue, and combinations thereof.

28. The slicing apparatus of claim 1, wherein the biological tissue is human allograft.

29. The slicing apparatus of claim 1, wherein the biological tissue is human dermis.

30. A method for substantially uniform thinning of biological tissue to a predefined thickness using a slicing apparatus, said slicing apparatus comprising:
a base structure;
a motor-driven rotary blade mounted on the base structure; and
a cutting plate with a plurality of height adjustments tubes to support the biological tissue to be thinned at a selected height in relation to the motor driven rotary blade, said method comprising:
setting the height of said cutting plate;
placing biological tissue to be thinned downward on said cutting plate;
rotating said rotary blade;
and moving the biological tissue to be thinned across said rotating rotary blade until the biological tissue is cut to the desired length.

31. The method of claim 30, wherein the biological tissue to be thinned is orientated on said cutting plate with edge guides.

32. The method of claim 30, wherein said cutting plate allows the biological tissue to be moved across said rotating rotary blade at a substantially uniform height throughout the entire pass of the material over the blade.

33. The method of claim 30, wherein the biological tissue to be thinned is manually moved across said rotating rotary blade.
